(19) United States
(12) Patent Application Publication
(10) Pub. No.: US 2015/0265468 A1
(43) Pub. Date: Sep. 24, 2015

(54) SURGICAL SYSTEM FOR OPENING THE LENS CAPSULE IN AN EYE

(71) Applicant: Carl Zeiss Meditec AG, Jena (DE)

(72) Inventors: Joachim Steffen, Westhausen (DE); Marco Wilzbach, Stuttgart (DE); Michael Stefan Rill, Jena (DE); Johannes Heinrich Kindt, Weimar (DE)

(21) Appl. No.: 14/664,888

(22) Filed: Mar. 22, 2015

Related U.S. Application Data
(60) Provisional application No. 61/968,748, filed on Mar. 21, 2014.

(51) Int. Cl. A61F 9/007; A61F 9/008

(52) U.S. Cl. CPC A61F 9/00754; A61F 9/0079; A61F 9/008; A61F 9/00834; A61F 2009/0087; A61F 2009/00889

(57) ABSTRACT

A surgical system for opening the lens capsule in an eye includes an application instrument introducible into the anterior chamber of the eye through an incision in the cornea and/or in the sclera. The application instrument has a contact body that is placeable against the anterior capsular wall of the lens capsule of the eye. Light provided by a light source for generating heat in a contact zone of the capsular wall contacting the contact body is appliable to the contact body. The heat has thermal energy that coagulates the biological tissue in the contact zone.

### CROSS REFERENCE TO RELATED APPLICATION

This application claims priority of U.S. provisional patent application Ser. No. 61/968,748, filed Mar. 21, 2014, the entire content of which is incorporated herein by reference.

### FIELD OF THE INVENTION

The invention relates to a surgical system for opening the lens capsule in an eye and includes an application instrument introducible into the anterior chamber of the eye through an incision in the cornea and/or in the sclera. Moreover, the invention also relates to a method for preparing an opening of the lens capsule in an eye.

### BACKGROUND OF THE INVENTION

Such a system and such a method are known from U.S. Pat. No. 8,591,577. There, it is proposed to embody an application tool arrangeable in the anterior chamber as a coil body which can be placed against the lens capsule and can be excited by means of a primary coil in order to coagulate the tissue of the capsular wall locally by means of heat.

Lenses of human eyes or eyes of mammals, which are occluded by a cataract, can be removed by a cataract operation referred to as phacoemulsification and can be replaced by intraocular lenses (IOLs). A necessary step during this operation lies in opening the anterior capsular bag of the corresponding eye by virtue of an opening being introduced into the anterior capsular wall of the lens capsule. During phacoemulsification, the occluded eye lens is comminuted through this opening by means of ultrasound and then removed. Thereupon, the intraocular lens (IOL) is introduced through this opening, the intraocular lens replacing the natural lens of the eye.

What is decisive for a successful phacoemulsification is that there are no complications when the anterior capsular bag is opened. In order to open the anterior capsular bag, the anterior capsular wall of the lens is generally pierced in the center by means of forceps or a needle tip, grasped by means of special forceps and then pulled clockwise or counterclockwise, with, in an ideal case, a largely round opening with a defined diameter being produced (capsulorhexis). On the one hand, the risk in this case is that the tear in the capsule is too big. By way of example, the tear can reach up to the lens equator. It is also possible for the posterior capsular wall to be destroyed during capsulorhexis, leading to the natural lens falling, that is, slipping into the vitreous humor. This is a treatment error which can only be corrected with great effort. This is because, in this case, the whole vitreous humor needs to be removed and replaced by a saline solution. Moreover, if the capsular bag is opened too far, the artificial intraocular lens does not have a good hold in the operated-on eye. The consequence of this is that the lens position can be displaced in an uncontrolled fashion during the healing process. This in turn can lead to a poor refraction result of the operated-on eye. On the other hand, an opening diameter which is too small can make the introduction of the artificial intraocular lens more difficult.

U.S. Pat. No. 8,562,596 has disclosed a laser cataract system in which a pulsed laser beam from a femtosecond laser is used to apply energy into the tissue of a patient eye locally on a micrometer scale. Here, the laser beam is guided along a provided cut line by quick, serial scanning. This method, which is also referred to as capsulotomy, is relatively time-consuming and therefore requires a rigid fixation of the eye of a patient, which is connected with much outlay. This method uses laser/tissue interactions such as photocoagulation, photoablation or photodisruption.

### SUMMARY OF THE INVENTION

It is an object of the invention to enable, with relatively little complexity, the precise opening of the lens capsule in the operated-on eye during a cataract surgical procedure.

The object is achieved by a system of the type set forth above, in which the application instrument has a contact body that is placeable against the anterior capsular wall of the lens capsule of the eye, with light provided by a light source for generating heat in a contact zone of the capsular wall contacting the contact body being appliable to the contact body, the heat having thermal energy that coagulates the biological tissue in the contact zone.

The concept underlying the invention is that the light of the light sources usually integrated into a surgical microscope can be used to coagulate the tissue of the capsular wall in a patient eye in a defined manner in order thus to open the lens capsule or weaken the tissue of the lens capsule in an exactly defined region such that the lens capsule can then be opened with forceps or another operation tool, without the lens capsule being exposed to excessive mechanical forces in the process.

The contact body of the application instrument preferably consists of a material at least partly absorbing the light from the light source. To this end, the material of the contact body can be stained by a dye, in particular by a dye from the group trypan blue (TP) or indocyanine green (ICG). A circular opening can be introduced into the lens capsule by the application instrument by virtue of the contact body having a ring-shaped form. Using such an application instrument, it is possible to prepare the lens capsule for a circular opening as well.

In a preferred embodiment of the surgical system, the application instrument has a cover skin secured at the contact body for at least partly covering the pupil of the eye. The cover skin has a surface preferably completely reflecting and/or completely scattering the light from the light source. By virtue of the contact body and the cover skin being flexible, the contact body can be folded with the cover skin such that the application instrument can be introduced through a narrow incision in the cornea or the sclera of the patient eye into the anterior chamber thereof.

The contact body can also consist of a material at least partly guiding the light. By way of example, the contact body can be embodied as a portion of an optical waveguide extending in the form of a loop. The portion of the optical waveguide extending in the form of a loop can have a mirrored surface with a slit-shaped light emergence opening formed on the side that is placeable against the anterior capsular wall.

The light source in the surgical system can be, for example, a laser or a thermal emitter, in particular a xenon lamp. Preferably, the surgical system comprises a surgical microscope for the magnified observation of an object range, in which the light provided by the light source illuminates the object region. The surgical system can also contain a device for feeding a fluid medium into the eye, in particular for feeding a viscoelastic and/or a dye and/or air.

According to the invention, the preparation of an opening of the lens capsule in an eye can be performed by the following steps: providing an application instrument with a contact body that is placeable against the anterior capsular wall of the lens capsule of the eye, providing a light source for applying light to the contact body, placing the contact body against the anterior capsular wall, applying light to the contact body and at least partly absorbing the light from the light source applied to the contact body in a dye. Here, according to the invention, the capsular wall can be stained by the dye. By virtue of forming an air bubble in the capsular wall, it is possible to arrange therein a contact body, which is preferably embodied as a loop-shaped optical waveguide and onto which light is applied, which contact body subjects the light guided therein to total internal reflection at the portions of the surface of the contact body not contacting the capsular wall and releases the light guided thereby into the capsular wall through the portions of the surface of the contact body contacting the capsular wall.

### BRIEF DESCRIPTION OF THE DRAWINGS

The invention will now be described with reference to the drawings wherein:

FIG. 1 shows a surgical system for opening the lens capsule in an eye, comprising a surgical microscope;

FIG. 2 shows an application instrument of the system;

FIG. 3 shows an application instrument placed against the anterior capsular wall of an eye;

FIG. 4 shows a partial view of the application instrument as a section;

FIG. 5 shows a further application instrument with an alternative setup for use in a surgical system for opening the lens capsule in an eye;

FIG. 6 shows a partial view of the further application instrument as a section;

FIG. 7 shows a further application instrument with an alternative setup for use in a surgical system for opening the lens capsule in an eye;

FIG. 8 shows a partial view of the further application instrument as a section;

FIG. 9 shows the further application instrument placed against the anterior capsular wall of an eye;

FIG. 10 shows a further application instrument with an alternative setup for use in a surgical system for opening the lens capsule in an eye; and,

FIG. 11 shows a device for providing a viscoelastic in the surgical system for opening the lens capsule in an eye.

### DESCRIPTION OF THE PREFERRED EMBODIMENTS OF THE INVENTION

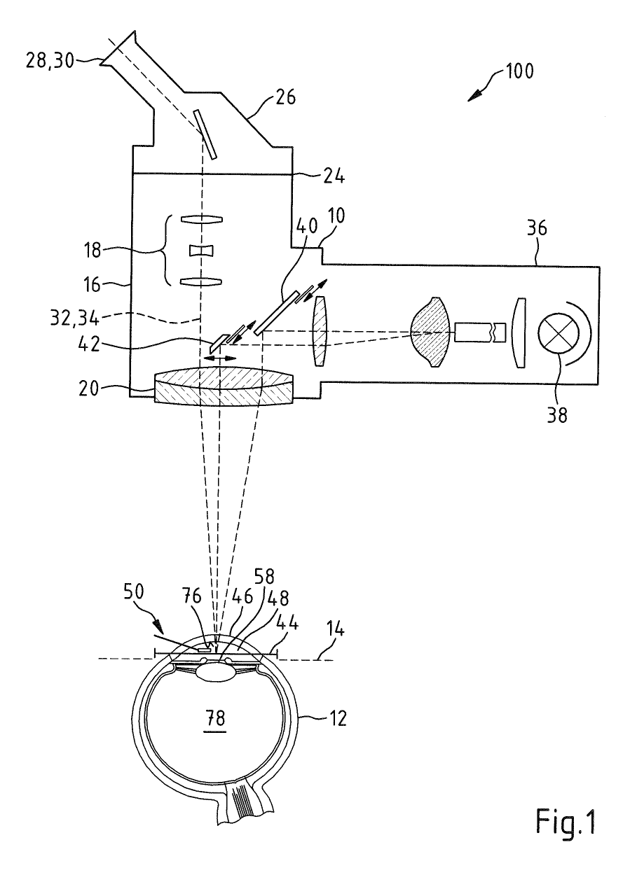

The surgical system 100 shown in FIG. 1 contains a surgical microscope 10 configured as an ophthalmic surgical microscope. The surgical microscope 10 serves for the magnified observation of a patient eye 12, which is arranged in an object region 14. The surgical microscope 10 has a surgical microscope main body 16 which holds imaging optics with a magnification system 18 and a microscope main objective system 20. It has a binocular tube 26 with a first and a second eyepiece (28, 30) for a left-hand and right-hand eye of an observing person, the binocular tube being connected to the main body 16 at an interface 24. The microscope main objective system 20 in the surgical microscope 10 is passed through by a first observation beam path 32 and a second observation beam path 34 from the object region 14.

The surgical microscope 10 contains an illumination system 36 with a xenon light source 38. The illumination system 36 has a first and a second illumination mirror (40, 42) arranged on the side of the microscope main objective system 20 facing away from the object region 14. The object region 14 of the surgical microscope 10 can be illuminated in an illuminated field 44 by the illumination system 36. Here, the illumination light provided by the xenon light source 38 has a wavelength spectrum similar to the natural sunlight in the visible spectral range. In the patient eye 12, the cornea 46 is opened by means of a narrow incision so that an application instrument 50 can be introduced into the anterior chamber 48.

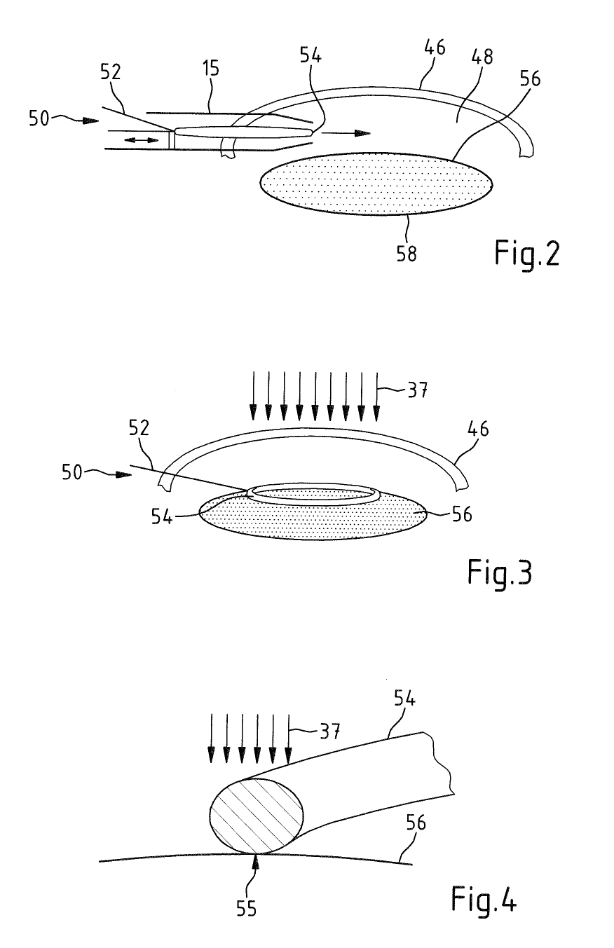

FIG. 2 shows the insertion of the application instrument 50 into the anterior chamber 48 of the patient eye 12 using an injector 15. The application instrument 50 is configured as a ring applicator, which has a holding section 52 with a foldable contact body 54. The contact body 54 is ring shaped and consists of a material on the basis of silicone, enriched with the trypan blue (TP) dye.

As a result of the material-provided elasticity thereof, the contact body 54 unfolds independently after the insertion through the incision in the anterior chamber 48 and assumes its ring shape.

FIG. 3 shows the contact body 54 introduced into the anterior chamber 48 of the patient eye 12 in its unfolded state. Here, the contact body 54 is placed against the anterior capsular wall 56 of the lens capsule 58 of the patient eye 12. When the illumination light 37 of the xenon light source 38 is applied to the object region 14, the portion of the light lying in the yellow spectral range is absorbed by the contact body 54 of the application instrument 50. In the process, heat is generated in the contact body 54. The contact body 54 is heated by the application of the illumination light of the illumination system 36 in the surgical microscope. As a result of contact with the contact body 54, this heat is transferred to the capsular wall 56. As a consequence, the tissue of the capsular wall 56 coagulates in a ring-shaped manner in the contact zone 55, shown in FIG. 4, which is touched by the contact body 54. As a result, an intended tear point is generated in the capsular wall 56 of the lens capsule 58. As an alternative to this, the tissue surrounded by the ring-shaped contact body 54 may also be separated from the capsular wall 56 as a result of the ring-shaped coagulation of the tissue of the capsular wall 56. It can then be removed through the incision in the cornea 46 with the aid of forceps.

Here, the capsular wall 56 of the lens capsule 58 tears precisely along the coagulated, ring-shaped line. In this manner, the opening in the capsular wall 56 is producible in a reproducible manner with high quality using this method. In particular, ring applicators with different diameters can be used for different dimensions of openings in the capsular wall 56.

It should be noted that the application instrument 50 described above can be configured as a sterilizable disposable application instrument or as a sterilizable reusable application instrument. In principle, the application instrument can be provided with a multiplicity of different diameters of the ring of the contact body 54. It should also be noted that the application instrument 50 can also be configured with an adjustable contact body, which can be mechanically adjusted to differently large openings in the capsular wall on the basis of patient data established pre-surgery.

Naturally, in place of the light from the xenon light source, it is also possible that the light from a laser is applied to the contact body 54. Moreover, it is possible that the material of the contact body 54 of the application instrument 50 with the indocyanine green (ICG) dye or provides a light source absorbing the light of a light source in the preferably visible spectral range.

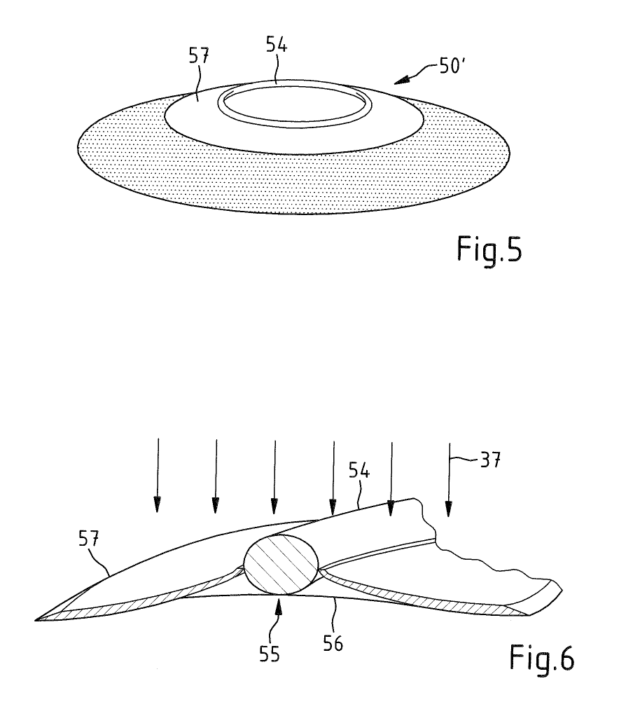

FIG. 5 shows a further application instrument 50′ embodied as a ring applicator, comprising a cover skin 57 acting as a cover shield. FIG. 6 shows a section of the application instrument 50′. The cover skin 56 prevents that the radiation from the xenon light source 38, which is required for heating the ring-shaped contact body 54 absorbing the light from the illumination system 36 of the surgical microscope 10, from reaching the retina 58 of the patient eye 12 and from possibly causing some damage there. The cover skin 57 adjoins the contact body 54 and consists of a material advantageously completely reflecting or scattering the light from the xenon light source 38.

The application instrument 50′ has a contact body 54, which absorbs the light lying in the yellow spectral range and therefore has a blue coloring. The cover skin 57 is white.

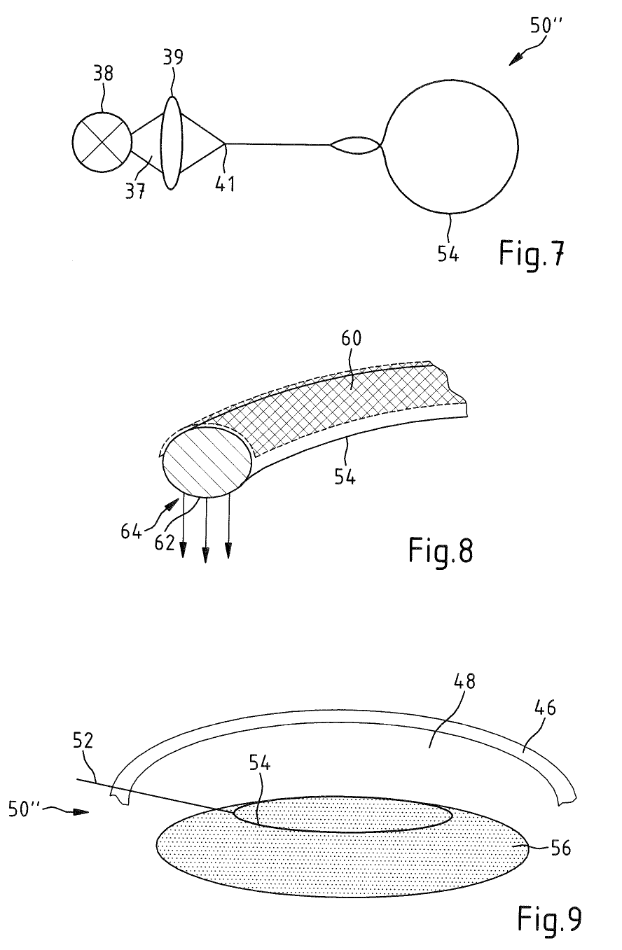

FIG. 7 shows a further application instrument 50″ in the form of an optical waveguide applicator. FIG. 8 shows a section of a partial view of the application instrument 50″. The application instrument 50″ has a contact body 54, which consists of a material at least partly conducting the light 37. Here, the contact body 54 is embodied as a loop made of an optical waveguide. In the application instrument 50″, light 37 is applied to the contact body 54 by virtue of the light from the light source 38 being coupled into an optical waveguide end 41 by means of a lens element 39.

The contact body 54 has a mirrored surface 60 with a slit-shaped light emergence opening 64 formed on the side 62 that is placeable against the anterior capsular wall 56. The application instrument 50″ also has a flexible configuration. Like the application instruments (50, 50′) described above, it can be introduced into the anterior chamber 48 of a patient eye 12 in a folded manner and there it can be placed against the capsular wall 56.

FIG. 9 shows the application instrument 50″ introduced into the anterior chamber 48 of a patient eye 12. In order to bring about the coagulation of the tissue of the capsular wall 56, it must in the present case be stained by a dye, for example, by the TP or ICG dye.

For the purposes of staining the capsular wall 56, the aforementioned dye must be injected into the patient eye 12. In order to prevent the injected dye from also staining the corneal endothelium 76 shown in FIG. 1, it is advantageous if, in a first step, air is injected into the anterior chamber 48 of the eye 12. In the patient eye 12 of a lying patient, the injected air in the form of air bubbles moves toward the cornea 46, and so the latter is protected the corneal endothelium 76 from unwanted staining during a subsequent injection of the dye. Using this measure, the selective staining of the tissue of the capsular wall 56 can be ensured when supplying dye into the patient eye 12.

Therefore, unlike the method known from U.S. Pat. No. 8,562,596 B2, the application tool 50″ also enables a locally defined application of laser radiation. Therefore, comparatively little radiation energy is applied to the patient eye 12 when the capsular wall 56 is opened.

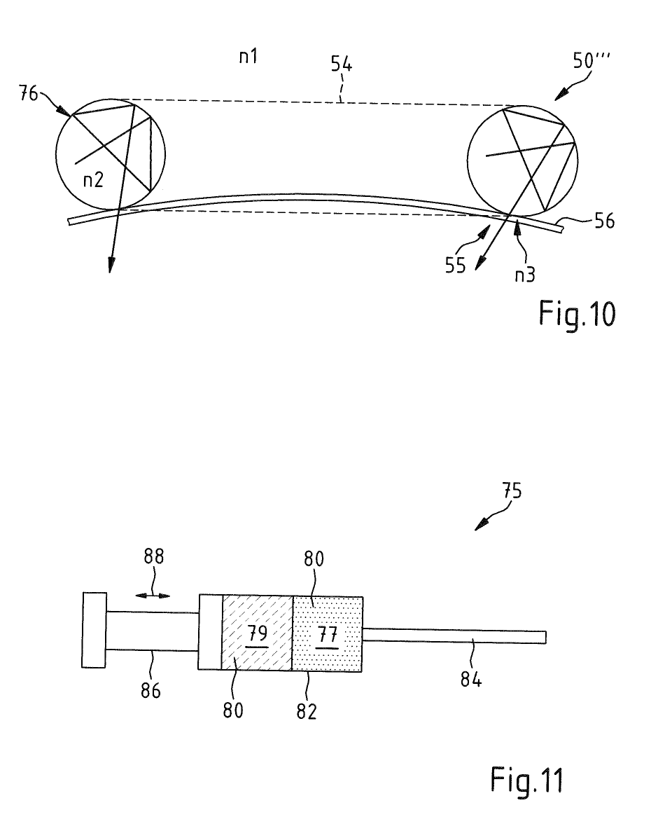

FIG. 10 shows a further application instrument 50′″, embodied as an optical waveguide applicator, with a contact body 54 embodied as a ring optical waveguide. In principle, the configuration of the application instrument 50′″ corresponds to the configuration of the application instrument 50″. The contact body 54 is not mirrored in the application instrument 50″. What is used in the application instrument 50′″ is that the light from a light source coupled into the contact body 54 experiences total internal reflection at the surface 76 of the contact body 54 if the refractive index n1 of the medium surrounding the contact body 54 and the refractive index n2 of the material of the contact body 54 guiding the light are sufficiently different. By contrast, the refractive index n2 of the material of the contact body 54 guiding the light corresponds to the refractive index n3=n2 of the biological tissue of the capsular wall 56.

In order to open the lens capsule 58, the contact body 54 of the application instrument 50′″ in the anterior chamber 48 is positioned against the capsular wall 56 in an air bubble introduced into the anterior chamber 48. As a consequence, the light applied to the contact body 54 reaches the contact zone 55 of the capsular wall 56 from the contact body 54 in the form of an evanescent light wave, but otherwise experiences total internal reflection at the portions of the surface 76 not contacting the contact zone 55.

The application instrument 50′″ enables very efficient introduction of light energy into the capsular wall 56 because the reflection losses R_{T} of the light at an optical interface in the case of total internal reflection are smaller than in the case of reflection R_{S} at a mirrored surface, that is, R_{T}<R_{S}, wherein R_{S}=90% is a typical value for R_{S}.

FIG. 11 shows a device 75, embodied as a syringe, for providing a viscoelastic (OVD) 77 and a dye 79 in a surgical system 100 for opening the lens capsule in a patient eye 12. The OVD is a viscous substance, which can be injected into the patient eye 12 in order to prevent the collapse of the eyeball connected with the emergence of vitreous humor from the vitreous chamber 78. The air and the OVD are held in the cylindrical cavity 80 of a cylinder 82. The cylindrical cavity 80 opens into an emergence channel 84. In the cylinder 82, a plunger 86 is guided such that it can undergo linear motion in the direction of the double-headed arrow 88.

The device 75 serves to initially inject the OVD and, thereupon, the dye into the anterior chamber 48 of the patient eye 12. Then, the viscoelastic forms a fluid protective layer on the corneal endothelium 76. What can be achieved by this is that the corneal endothelium 76 is not stained when the dye is injected, and so there is no risk that light radiation, for example, laser radiation, is absorbed there with heat being developed.

What can be achieved in the case of a surgical system configured for opening the lens capsule 58 in a patient eye 12 and containing the device 75 is that a surgeon need not use two different syringes for the supply of OVD and dye during a cataract operation. This enables more efficient progress of the cataract operation and reduces the risk of complications because, for example, this can prevent an inadvertent omission of the supply of dye.

It should be noted that the dye required for staining the capsular wall 56 can be supplied by a phacoemulsification handpiece of a phacoemulsification machine or an irrigator, like a BSS solution or silicone oil supplied to a patient eye 12. What this renders possible for the application of the dye is that a surgeon no longer needs to put to one side the irrigator or the phacoemulsification handpiece in order to pick up a syringe. The time required for a cataract operation can also be significantly reduced by this measure. It should be noted that further control mechanisms can also be brought to bear by linking the dye injection to the phacoemulsification machine. By way of example, provision can be made for a timer integrated into a phacoemulsification machine to measure how long a dye is situated in a patient eye in order then to rinse this dye after an ideal application time. Moreover, it is also advantageous that if the corneal endothelium was accidentally stained, which can be identified by means of, for example, a surgical microscope, a de-staining fluid can preferably be introduced into the patient eye automatically.

In conclusion, the following preferred features of the invention should, in particular, be registered: A surgical system 100 for opening the lens capsule 58 in an eye 12 includes an application instrument (50, 50′, 50″, 50′″) introducible into the anterior chamber 48 of the eye 12 through an incision in the cornea 46 and/or in the sclera 47. The application instrument (50, 50′, 50″, 50′″) has a contact body 54 that is placeable against the anterior capsular wall 56 of the lens capsule 58 of the eye 12. Light provided by a light source 38 for generating heat in a contact zone 55 of the capsular wall 56 contacting the contact body 54 is appliable to the contact body 54, the heat having thermal energy that coagulates the biological tissue in the contact zone 55.

It is understood that the foregoing description is that of the preferred embodiments of the invention and that various changes and modifications may be made thereto without departing from the spirit and scope of the invention as defined in the appended claims.

### LIST OF REFERENCE SIGNS

10 Surgical microscope
12 Patient eye
14 Object region
15 Injector
16 Surgical microscope main body
18 Imaging optics
20 Microscope main objective system
22 Main body
24 Interface
26 Binocular tube
28, 30 Eyepiece
32, 34 Observation beam path
36 Illumination system
37 Illumination light
38 Xenon light source
39 Lens element
41 Optical waveguide end
40, 42 Illumination mirror
44 Illuminated field
46 Cornea
48 Anterior chamber
50, 50′, 50″, 50′″ Application instrument
52 Holding section
54 Contact body
55 Contact zone
56 Capsular wall
57 Cover skin
58 Lens capsule
59 Retina
60 Surface
62 Side
64 Light emergence opening
66 Cover skin
68 Pupil
70 Surface
72 Optical waveguide
74 Mirrored surface
75 Device
76 Corneal endothelium
77 Viscoelastic
78 Vitreous chamber
79 Dye
80 Cavity
82 Cylinder
84 Emergence channel
86 Plunger
88 Double-headed arrow
100 System

What is claimed is:

1. A surgical system for opening the lens capsule in an eye, the surgical system comprising:
an application instrument which can be introduced into the anterior chamber of the eye through an incision in at least one of the cornea and sclera;
said application instrument including a contact body configured to be placeable upon the anterior capsular wall of the lens capsule of the eye;
said contact body and the anterior capsular wall conjointly defining an interface whereat a contact zone of the anterior capsular wall touches said contact body; and,
a light source configured for providing light to said contact body to generate heat in said contact zone to apply thermal energy to coagulate the biological tissue in said contact zone.

2. The surgical system of claim 1, wherein said contact body is made of a material which at least partially absorbs said light of said light source.

3. The surgical system of claim 2, wherein said material is stained with a dye.

4. The surgical system of claim 3, wherein said dye is selected from a group including trypan blue or indocyanine green (ICG).

5. The surgical system of claim 1, wherein said contact body is configured to have an annular shape.

6. The surgical system of claim 1, wherein said application instrument includes a cover skin fixed to said contact body; said cover skin is configured to at least partially cover the pupil of the eye and said cover skin has a surface reflecting and/or scattering the light of said light source.

7. The surgical system of claim 6, wherein said cover skin is flexible.

8. The surgical system of claim 1, wherein said contact body is made of a material at least partly conducting said light.

9. The surgical system of claim 8, wherein said contact body is configured as a segment of a light conductor extending in the form of a loop.

10. The surgical system of claim 9, wherein said segment of said light conductor has a mirrored surface and has a side placeable upon the anterior capsular wall; and, said side has a light outlet configured to have a slit-like shape.

11. The surgical system of claim 1, wherein said contact body is flexible.

12. The surgical system of claim 1, wherein said light source is a laser or a thermal radiator.

13. The surgical system of claim 12, wherein said thermal radiator is a xenon lamp.

14. The surgical system of claim 12, further comprising:
a surgical microscope for viewing an object region;
said light source being configured to illuminate said object region; and,
a device for metering a fluid medium into the eye.

15. The surgical system of claim 14, wherein said fluid medium is at least one of the following: viscoelastikum, dye and air.

16. The surgical system of claim 12, further comprising:
a surgical microscope for viewing an object region; and,
said light source being configured to illuminate said object region.

17. The surgical system of claim 12, further comprising:
a surgical microscope for viewing an object region; and,
a device for metering a fluid medium into the eye.

18. The surgical system of claim 17, wherein said fluid medium is at least one of the following: viscoelastikum, dye and air.

19. A method for preparing an opening of the lens capsule in an eye, the method comprising the steps of:
providing an application instrument having a contact body placeable on the anterior capsular wall of the lens capsule of the eye;
providing a light source for applying light to the contact body;
placing the contact body on the anterior capsular wall;
applying light to the contact body; and,
at least partly absorbing the light from the light source applied to the contact body in a dye.

20. The method of claim 19, further comprising the step of staining the capsular wall with the dye.

21. The method of claim 19, further comprising the step of generating an air bubble formed ahead of the capsular wall, in which air bubble the contact body is arranged while releasing light entering into the capsular wall.
